(19) United States
(12) Patent Application Publication
(10) Pub. No.: US 2018/0021467 A1
(43) Pub. Date: Jan. 25, 2018

(54) METHOD OF BLOCKING PERCEPTION OF MALODOR

(71) Applicant: Monell Chemical Senses Center, Philadalephia, PA (US)

(72) Inventors: Joel Drewery Mainland, Philadalephia, PA (US); Paul Wise, Downingtown, PA (US)

(21) Appl. No.: 15/547,916

(22) PCT Filed: Feb. 4, 2016
(86) PCT No.: PCT/US2016/016493

§ 371 Date: Aug. 1, 2017

Related U.S. Application Data
(60) Provisional application No. 62/112,379, filed on Feb. 5, 2015.

(51) Int. Cl. A61L 9/01; G01N 33/50

(52) U.S. Cl. CPC A61L 9/01; G01N 33/5041; G01N 33/5023; G01N 2333/705

(57) ABSTRACT

Methods and compositions for blocking, inhibiting or reducing the perception of malodor by sensory receptors of a mammalian subject comprise an effective amount of a compound of Table I, a derivative thereof or any combination or mixture of such compounds. Methods for screening for such compositions in cell-based assays are provided.

### BACKGROUND OF THE INVENTION

Detection of malodors, i.e., natural and synthetic compounds and compositions which are in a variety of states, liquid, solid, emulsions, etc. and which emit an unpleasant odor, shape a large part of the olfactory experience of humans and therefore impact the quality of life. The traditional approach to blocking perception of malodors involves masking a malodor with other, typically multiple, odorous compounds. Masking may operate at the periphery on olfactory receptors and/or at more central levels in the brain. It is difficult to identify the biological mechanisms of action for masking and thus difficult to systematically develop maskers.

The mammalian olfactory system detects odors using a large repertoire of canonical olfactory receptors (ORs) of which there are greater than 400 in human, as well as a small repertoire of recently described trace amine-associated receptors (TAARs). As with ORs, TAARs are expressed in the olfactory epithelium and each TAAR allele defines a population of neurons that do not express another OR or TAAR. Although several TAARs have been shown to respond to amines, little is known about the role of TAARs in olfactory perception and behavior. Some early studies in mice suggest that deletion of a single TAAR can have a large influence on perception of the receptor's agonist. A study describing TAAR expression in the human olfactory epithelium (OE) reported detection of mainly hTAAR5, to a lesser extend hTAAR8 and at lower levels all other hTAAR genes as well (Carnicelli V, et al. 2010 Chemosensory Perception 3: 99-107). With the exception of hTAAR1, all human TAAR cDNAs were detected exclusively in OMP-positive nasal biopsies, an indication for a specific OE expression.

Wallrabenstein, I, et al, 2013, Plos One, 8(2):e54950 described a ligand screening for human TAAR5, the TAAR subtype with the highest expression level in human OE. Human TAAR5 can be activated in a concentration-dependent manner by trimethylamine (TMA) and with less efficacy by dimethylethylamine. However, Wallrabenstein demonstrated that human TAAR5 is relatively specific—as 42 amines with high structural similarity to TMA and a number of tested mixtures failed to activate the receptor.

There is a need in this field for methods and compositions that can be employed to inhibit, reduce or block the perception of malodors by the TAARs.

### SUMMARY OF THE INVENTION

As described herein, methods and compositions are described for identifying new and known compounds that act as antagonists to malodors and that directly target receptors in the nasal cavity and using such compounds in admixture with malodorous compositions and fragrances to alter or reduce their perception when they are inhaled. Reliable methods are described for identifying effective antagonists to any given malodor and validating and refining them at the cellular and psychophysical levels. Studies of human genetic variation show that humans lacking a functional allele of a given OR perceive the ligand of the OR to be less intense than subjects with a functional allele. Thus, identifying an antagonist for a given receptor similarly reduces the intensity of the receptor's ligand.

In one aspect, the invention provides a composition for blocking, inhibiting or reducing the perception of malodor by sensory receptors of a mammalian subject, the composition comprising an effective amount of a compound of Table I or a derivative thereof and a carrier.

In another aspect, a method for blocking, inhibiting or reducing the perception of malodor by a mammalian subject comprises blocking the TAAR olfactory receptors with a compound of Table I or a derivative thereof. In one embodiment, the “blocking” involves applying the compounds or a composition containing the compound to a substrate on which the malodor is present. In another embodiment, the “blocking” involves dispersing the compounds or a composition containing the compound into the air space in which the malodor is present. In still another embodiment, the “blocking” is accomplished by admixing the compound into a composition for ingestion which contains a malodorous substance.

In still another aspect, a method for identifying or screening malodor blocking molecules, compounds or compositions is provided. The method involves screening for the effect of a test molecule on the expression or activity of a trace amine-associated receptor (TAAR) protein in a cell-based assay. In one embodiment, this method involves contacting a test molecule with a mammalian cell or cell line that expresses a TAAR protein, i.e., the TAAR5 protein, under in vitro culture conditions; and measuring the expression level or functional activity of the TAAR by the contacted cell or cell line in both the presence and absence of the test compound and/or a control. In one embodiment, binding of the TAAR protein by a test molecule can inhibit, reduce or block malodor perception. Thus, binding is an indication of a malodor blocker or reducer. In another embodiment, a decrease in protein expression or functional activity of the TAAR protein by the cell or cell line contacted with the test molecule over that of a positive or negative control cell or cell line identifies a test molecule that can inhibit, reduce or block malodor perception. In still another embodiment, the displacement of the control molecule, which is known to bind or interact with the TAAR protein, is an indication that the compound is a malodor blocker.

Other aspects and advantages of the invention will be readily apparent from the following detailed description of the invention.

### BRIEF DESCRIPTION OF THE DRAWINGS

FIG. 1A is a graph showing dose response of the binding of trimethylamine (TMA) to the receptor TAAR5 in the absence (▪) or presence at 5 mM () of the antagonist phenethyl benzoate. Luciferase activity shows the activation of the trace amine-associated receptor.

FIG. 1B is a graph showing dose response of the binding of TMA to the receptor TAAR5 in the absence (▪) or presence at 5 mM () of the antagonist trans-2-nonen-1-ol. Luciferase activity showed the activation of the trace amine-associated receptor.

FIG. 1C is a graph showing dose response of the binding of TMA to the receptor TAAR5 in the absence (▪) or presence at 5 mM () of the antagonist isoeugenyl phenylacetate. Luciferase activity showed the activation of the trace amine-associated receptor.

FIG. 1D is a graph showing dose response of the binding of TMA to the receptor TAAR5 in the absence (▪) or presence at 5 mM () of the antagonist tributyl-2-acetylcitrate. Luciferase activity showed the activation of the trace amine-associated receptor.

FIG. 1E is a graph showing dose response of the binding of TMA to the receptor TAAR5 in the absence (▪) or presence at 5 mM () of the antagonist bis(2-methyl-3-furyl) disulfide. Luciferase activity showed the activation of the trace amine-associated receptor.

FIG. 2A is a graph showing dose response of the binding of TMA to the receptor TAAR5 in the absence (▾) or presence at 50.11 μM () or 251.19 μM (▪), or 478.63 μM (▴) of the antagonist phenethyl benzoate. Luciferase activity showed the activation of the trace amine-associated receptor.

FIG. 2B is a graph showing dose response of the binding of TMA to the receptor TAAR5 in the absence (▾) or presence at 100 μM () or 316.22 μM (▪), or 703.07 μM (▴) of the antagonist trans-2-nonen-1-ol. Luciferase activity showed the activation of the trace amine-associated receptor.

FIG. 2C is a graph showing dose response of the binding of TMA to the receptor TAAR5 in the absence (▾) or presence at 100 μM () or 316.22 μM (▪), or 703.07 μM (▴) of the antagonist isoeugenyl phenylacetate. Luciferase activity showed the activation of the trace amine-associated receptor.

FIG. 2D is a graph showing dose response of the binding of TMA to the receptor TAAR5 in the absence (▾) or presence at 74.14 μM () or 194.98 μM (▪), or 241.55 μM (▴) of the antagonist tributyl-2-acetylcitrate. Luciferase activity showed the activation of the trace amine-associated receptor.

FIG. 2E is a graph showing dose response of the binding of TMA to the receptor TAAR5 in the absence (▾) or presence at 31.62 μM () or 77.62 μM (▪), or 157.67 μM (▴) of the antagonist bis(2-methyl-3-furyl-disulfide). Luciferase activity showed the activation of the trace amine-associated receptor.

FIG. 3 shows the inhibition percentage plot from the original antagonist screen with the five antagonists confirmed in the dose-response as dark spots appearing above the horizontal line that represents 80% inhibition.

FIG. 4A is a graph showing the inhibition of luciferase activity by increasing concentrations of test odors. Cells were transfected with TAAR5 and stimulated with 500 μM TMA (▾) or were transfected with a vector control and stimulated with 1 ump forskolin (▪) to screen for nonspecific inhibition. FIG. 4A shows the response to the antagonist isoeugenyl phenylacetate.

FIG. 4B is a graph similar to that of FIG. 4A, showing the response to a physicochemically similar odorant, Famotidine.

FIG. 4C is a graph similar to that of FIG. 4A, showing the response to a physicochemically similar odorant, Argumea BHT.

FIG. 4D is a graph similar to that of FIG. 4A, showing the response to a physicochemically similar odorant, Sulpiride.

FIG. 4E is a graph similar to that of FIG. 4A, showing the response to a physicochemically similar odorant, Phytol.

FIG. 4F is a graph similar to that of FIG. 4A, showing the response to a physicochemically similar odorant, Delta-tetradecalactone.

FIG. 4G is a graph similar to that of FIG. 4A, showing the response to a physicochemically similar odorant, Piperine.

FIG. 4H is a graph similar to that of FIG. 4A, showing the response to a physicochemically similar odorant, Acetohexamide.

FIG. 5A is a graph showing human perceptual ratings of “fishiness” for multiple concentrations of TMA either alone, or when combined with trans-2-nonen-1-ol, or a control odor (linalool). Antagonist trans-2-nonen-1-ol decreases fishy intensity, but control odor Linalool does not.

FIG. 5B is a graph showing human perceptual ratings of “fishiness” for multiple concentrations of TMA either alone, or when combined with isoeugenyl phenylacetate, or a control odor (linalool). Antagonist isoeugenyl phenylacetate decreases fishy intensity, but control odor Linalool does not.

FIG. 6. is a diagram explaining the experimental setup for making dichorhinic and physical mixtures.

FIG. 7 is a graph summarizing the data acquired as described in Example 2 for evaluating a TAAR antagonist by human psychophysical response in a split-nostril design. The graph shows ratings with either an antagonist (trans-2-nonen-1-ol) or the control odor (linalool). The antagonist had a larger peripheral component of mixture suppression than the control odorant linalool.

### DETAILED DESCRIPTION OF THE INVENTION

The methods and compositions described herein employ certain compounds that reduce or counteract the effects of malodor molecules by competitively binding to the same olfactory receptors, namely the trace amine-associated receptors TAARs.

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein. By “mammalian subject” is meant primarily a human, but also domestic animals, e.g., dogs, cats; and livestock, such as cattle, pigs, etc.; common laboratory mammals, such as primates, rabbits, and rodents; and pest or wild animals, such as deer, rodents, rabbits, squirrels, etc. The terms “a” or “an” refers to one or more, for example, “an assay” is understood to represent one or more assays. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

### I. TAAR ANTAGONIST COMPOUNDS

“TAAR” refers to a family of trace amine-associated receptors that are G-protein coupled receptors (GPCR) and have been identified as a second class of olfactory receptors in the olfactory epithelia of certain vertebrates, including humans. The TAARs are particularly sensitive to amines, which are largely found to be aversive by many species, including humans. Amines such as putricine and cadaverine signal decay in animals and other alkyl amides signal spoilage in food. In humans 6 functional TAARs have been identified, i.e. TAAR1 (Swiss Prot/Ref Seq Q96RJ0/NP_612200), TAAR9 (Swiss Prot/Ref Seq Q96R19/NP_778227), TAAR6 (Swiss Prot/Ref Seq Q96R18/NP_778237), TAAR8 (Swiss Prot/Ref Seq Q969N4/NP_444508), TAAR2 (Swiss Prot/Ref Seq Q9PIP5/NP_055441), and TAAR5 (Swiss Prot/Ref Seq O14804/NP_003958).

The “TAAR antagonist compounds” as described herein include in one embodiment, a compound identified in Table I, including phenethyl benzoate, phenethyl anthranilate, trans-2-nonen-1-ol, isoeugenyl phenylacetate, phenethyl phenylacetate, bis(2-methyl-3-furyl) disulfide, tributyl-2-acetylcitrate, lauric acid, phenethylsalicylate, benzyl benzoate, geranyl alcohol, thibetolide, benzyl disulfide, aurantiol, isoambrettolide, allyl isothiocyanate, 5-cyclohexadecen-1-one, androstadienone, alpha-amylcinnamaldehyde, benzyl mercaptan, benzyl salicylate,nabumetone, 1,3-diphenyl-2-propanone, benzyl phenylacetate, ethyl maltol, 3-decen-2-one, trans, trans-2,6-nonadienal, galaxolide, isocyclemone E, hexanol, 2-phenyl-2-butenal, 2,5-dihydroxy-1,4-dithiane, 2-decenal, cycloheptanecarbaldehyde, tobacco absolute, isobutyl benzoate, benzyl alcohol, cis-4-heptenal, florhydral, phenethyl 2-furoate, myristic acid, 3,7-dimethyl-1-octanol, alpha cedrene, 3-(methylthio)propionaldehyde, 1-tetradecanol, carvacrol, 2-furanmethanethiol formate, 2-methyl-3-(3,4-methylenedioxyphenyl)-propanal. The terms “compound”, “composition”, “reagent” or “molecule” as used herein may be used interchangeably.

In another embodiment, the compounds which operate as antagonists of the TAARs are mixtures of two or more of the compounds identified in Table I below. Table I lists by chemical name, CAS#, and structure certain compounds and natural or synthetic mixtures of compounds that inhibit receptor binding between TMA and TAAR5.

TABLE I

| Chemical | | | Percent | |
|---|---|---|---|---|
| Name | CAS# | SMILES | Inhibition | Structure |
| Phen- ethyl benzoate | 94-47-3 | C(C1═CC═CC═ C1)(═O)OCCC 2═CC═CC═C2 | 93.49413573 | 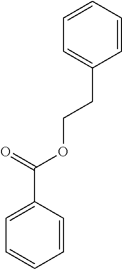 [SMILES: O=C(OCCc1ccccc1)c1ccccc1] |
| Phen- ethyl anthra- nilate | 133-18-6 | C(C1═CC═CC═ C1N)(═O)OCC C2═CC═CC═C2 | 92.79782596 | 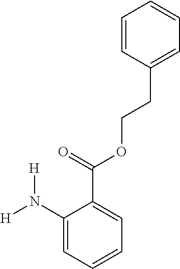 [SMILES: Nc1ccccc1C(=O)OCCc1ccccc1] |
| Trans-2- Nonen-1- ol | 31502-14-4 | CCCCCCC═CCO | 89.3243314 | 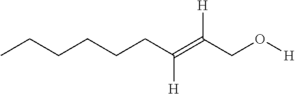 |
| Isoeu- genyl phenyl- acetate | 120-24-1 | C1═CC(═CC(═C 1OC(CC2═CC═ CC═C2)═O)OC)\ C═C\C | 89.10893266 | 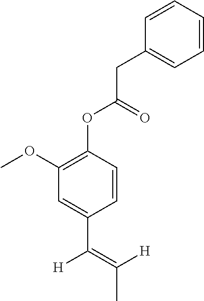 [SMILES: C/C=C/c1ccc(OC(=O)Cc2ccccc2)c(OC)c1] |
| Phen- ethyl phenyl- acetate | 102-20-5 | C1(═CC═CC═C1) CC(═O)OCC C2═CC═CC═C2 | 88.93408415 | 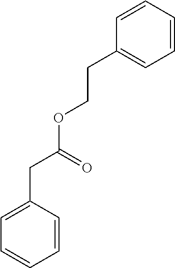 [SMILES: O=C(Cc1ccccc1)OCCc1ccccc1] |
| Bis(2- Methyl- 3-furyl) disulfide | 28588-75-2 | CC1═C(C═CO1) SSC2═C(OC═C2)C | 88.30521529 | 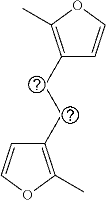 |
| Tributyl- 2-Acetyl- citrate | 77-90-7 | C(CCC)OC(C(C (O)(C(═O)OCC CC)CC(═O)OC CCC)C(C)═O)═O | 87.83159494 | 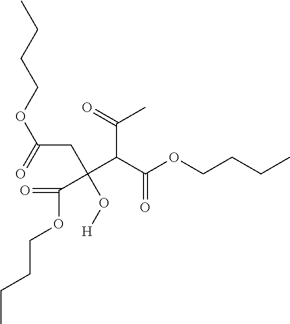 [SMILES: CCCCOC(=O)CC(O)(C(=O)OCCCC)C(C(C)=O)C(=O)OCCCC] |
| Lauric acid | 143-07-7 | C(CCCCCCCCCCC) (═O)O | 86.02191042 | 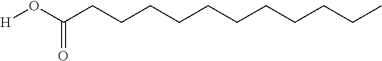 |
| Phen- ethyl salicylate | 87-22-9 | C(C1═CC═CC═ C1O)(═O)OCCC2═ CC═CC═C2 | 85.03690353 | 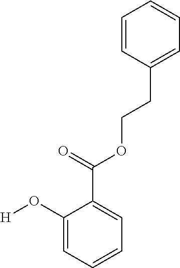 [SMILES: O=C(OCCc1ccccc1)c1ccccc1O] |
| Benzyl benzoate | 120-51-4 | C1═CC═C(C═C1) COC(═O)C2═ CC═CC═C2 | 83.86866071 | 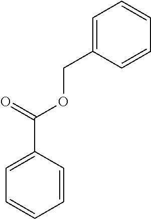 [SMILES: O=C(OCc1ccccc1)c1ccccc1] |
| geraniol; geranyl alcohol | 106-24-1 | CC(═CCCC(═ CCO)C)C | 81.25493737 | 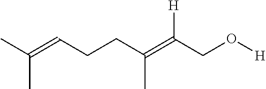 |
| Thibeto- lide | 106-02-5 | C1CCCCCCCO C(═O)CCCCCC1 | 81.02205346 | 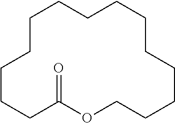 [SMILES: O=C1CCCCCCCCCCCCCCO1] |
| Benzyl disulfide | 150-60-7 | C(C1═CC═CC═ C1)SSCC2═CC═ CC═C2 | 80.99795602 | 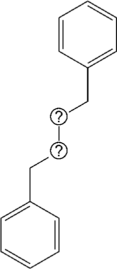 [SMILES: c1ccc(CCCCc2ccccc2)cc1] |
| aurantiol | 89-43-0 | CC(CCCC(C)(C) O)CC═NC1═CC═ CC═C1C(═O) OC | 79.59925029 | 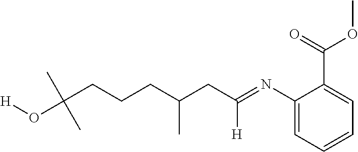 [SMILES: COC(=O)c1ccccc1/N=C/CC(C)CCCC(C)(C)O] |
| Isoam- bretto- lide | 28645-51-4 | C1CCCC═CCCC CCCOC(═O)CCC1 | 78.69039625 | 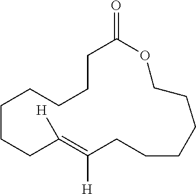 [SMILES: O=C1CCCCCCC/C=C/CCCCCCO1] |
| Allyl isothio- cyanate | 57-06-7 | C═CCN═C═S | 77.39469277 | 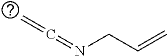 |
| velvione; 5-Cyclo- hexa- decen-1- one | 37609-25-9 | C1CCCCCC(═O) CCCC═CCCCC1 | 76.04597756 | 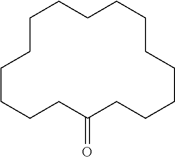 [SMILES: O=C1CCCCCCCCCCCCCCC1] |
| Androsta dienone; 4,16- Androsta- dien-3- one; Androsta- 4,16- dien-3- one | | C[C@]12CC[C@H] 3[C@H] ([C@@H]1CC═ C2)CCC4═CC(═ O)CC[C@]34C | 76.03898023 | 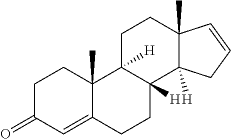 |
| Alpha- Amyl- cinna- malde- hyde | 122-40-7 | C(CCCC)C(C═O)═ CC1═CC═CC═C1 | 75.82559671 | 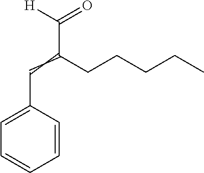 |
| Benzyl mercap- tan | 100-53-8 | C(C1═CC═CC═C1)S | 75.82477424 | 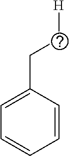 |
| Benzyl salicylate | 118-58-1 | C(C1═CC═CC═ C1O)(═O)OCC2═ CC═CC═C2 | 74.175484 | 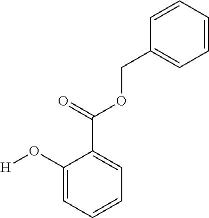 [SMILES: O=C(OCc1ccccc1)c1ccccc1O] |
| Nabu- metone | 42924-53-8 | CC(═O)CCC1═ CC2═C(C═C1)C═ C(C═C2)OC | 73.81349512 | 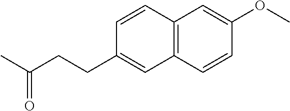 [SMILES: COc1ccc2cc(CCC(C)=O)ccc2c1] |
| 1,3- Diphenyl- 2-propa- none; 1,3- diphenyl propan- 2-one | 102-04-5 | C1(═CC═CC═C1) CC(CC2═CC═ CC═C2)═O | 73.07735097 | 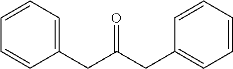 [SMILES: O=C(Cc1ccccc1)Cc1ccccc1] |
| Benzyl phenyl- acetate | 102-16-9 | C1(═CC═CC═C1) CC(═O)OCC2═ CC═CC═C2 | 72.74868031 | 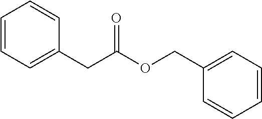 [SMILES: O=C(Cc1ccccc1)OCc1ccccc1] |
| ethyl maltol; 2- Ethyl-3- hydroxy- 4H- pyran-4- one; | 4940-11-8 | CCC1═C(C(═O) C═CO1)O | 72.48750283 | 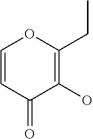 |
| 3-Decen- 2-one | 10519-33-2 | CC(C═CCCCCCC)═O | 72.48320563 | 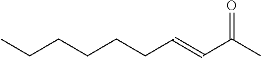 |
| Trans, trans-2,6- Nona- dienal | 17587-33-6 | CCC═CCCC═CC═O | 71.43782119 | 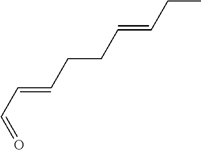 |
| Galaxo- lide; Cyclo- penta[g]- 2-benzo- pyran | 1222-05-5 | CC1COCC2═C C3═C(C═C12)C (C(C3(C)C)C)(C)C | 71.22506603 | 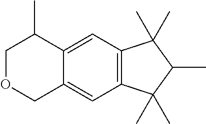 [SMILES: CC1COCc2cc3c(cc21)C(C)(C)C(C)C3(C)C] |
| iso E super; isocycle- mone E | 54464-57-2 | CC1CC2═C(CC 1(C)C(═O)C)C (CCC2)(C)C | 69.59631664 | 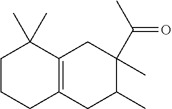 [SMILES: CC(=O)C1(C)CC2=C(CCCC2(C)C)CC1C] |
| Hexanal | 66-25-1 | C(CCCCC)═O | 69.41130111 | 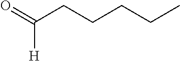 |
| 2-Phenyl- 2-butenal | 4411-89-6 | C1(═CC═CC═C1) C(C═O)═CC | 68.85203259 | 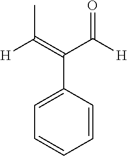 |
| 2,5- Dihydro- xy-1,4- dithiane | 40018-26-6 | OC1CSC(CS1)O | 67.8995137 | 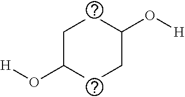 |
| 2-decenal | 3913-71-1 | CCCCCCCC═CC═O | 66.41225676 | 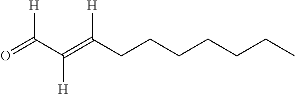 |
| Cyclohep- tane- carbal- dehyde | 4277-29-6 | C1CCCC(CC1)C═O | 64.40892353 | 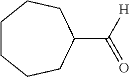 |
| tobacco absolute | 8037-19-2 | N/A-natural extract from Nicotiana tabacum | 63.92680009 | N/A |
| Isobutyl benzoate | 120-50-3 | C(C1═CC═CC═ C1)(═O)OCC(C)C | 63.29376005 | 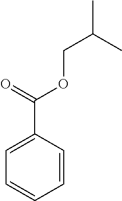 |
| benzyl alcohol | 100-51-6 | C(C1═CC═CC═C1)O | 62.53524633 | 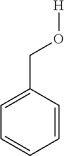 |
| cis-4- Heptenal | 6728-31-0 | C(CC\C═C/CC)═O | 61.45100612 | 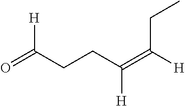 |
| florhydral | 125109-85-5 | CC(C)C1═CC(═ CC═C1)C(C)CC═O | 61.24846355 | 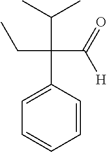 |
| TetraFin ® fish food (Tetra GmbH) | | N/A | 60.16814284 | N/A |
| ylang ylang | 8006-81-3 | N/A-natural oil | 60.00702521 | N/A |
| Phen- ethyl 2- furoate | 7149-32-8 | O1C═CC═C1C(═ O)OCCC2═CC═ CC═C2 | 59.59237137 | 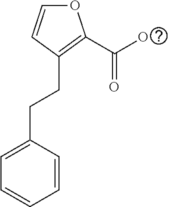 [SMILES: O=C(O)c1occc1CCc1ccccc1] |
| myristic acid | 544-63-8 | CCCCCCCCCC CCCC(═O)O | 59.08902049 | 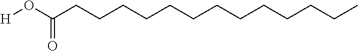 [SMILES: CCCCCCCCCCCCCC(=O)O] |
| 3,7- Dimethyl- 1-octanol | 106-21-8 | CC(CCO)CCCC (C)C | 57.09552513 | 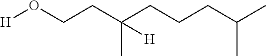 |
| vetiver indonesia | 8016-96-4 | N/A-Crude oil extracted from roots of Vetiver plant | 56.41059255 | N/A |
| (-)-α- Cedrene | 469-61-4 | CC1CCC2C13C C═C(C(C3)C2 (C)C)C | 56.12576296 | 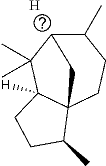 |
| 3- (Methyl- thio)pro- pional- dehyde | 3268-49-3 | CSCCC═O | 54.51807085 | 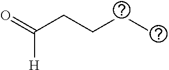 |
| 1-Tetra- decanol | 112-72-1 | CCCCCCCCCC CCCCO | 54.12176709 | 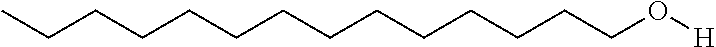 |
| Carvacrol | 499-75-2 | C1═C(O)C(═CC═ C1C(C)C)C | 52.161132 | 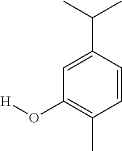 |
| 2-Furan- methane- thiol formate | 59020-90-5 | C(═O)O•O1C═ CC═C1CS | 50.39565655 | 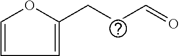 |
| 2- Methyl- 3-(3,4- methyl- enedioxy phenyl)- propanal | 1205-17-0 | CC(CC1═CC2═ C(C═C1)OCO2) C═O | 50.14512195 | 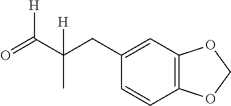 |

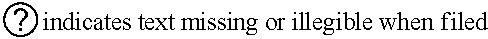

In other embodiments, the compounds which operate as antagonists of the TAARs are mixtures of racemates of one or more of the appropriate compounds of Table I. In still another embodiment, a compound which operates as antagonists of the TAARs is an enantiomer of a compound of Table I. In still another embodiment, a compound which operates as an antagonist of the TAARs is a derivative of a compound of Table I. In still another embodiment, a compound which operates as an antagonist of the TAARs is a combination of derivatives of the same compound of Table I. In still another embodiment, a compound which operates as an antagonist of the TAARs is a combination of derivatives of different compounds of Table I.

By “derivative” of a TAAR antagonist compound is meant a compound in which one of the carbon atoms in the straight chain molecule or in a heterocycle of a compound of Table I is optionally substituted with another chemical substituent. The optional substitution can occur by breaking a double bond in the heterocycle to enable addition of the new substituent.

The term “optionally substituted” as used herein refers to the base group having one or more substituents including, without limitation, H, halogen, CN, OH, NO₂, amino, alkyl, cycloalkyl, alkenyl, alkynyl, aryl, heterocyclic, heteroaryl, alkoxy, aryloxy, alkylcarbonyl, alkylcarboxy, arylthio, alkylamino, or —SO₂-(optionally substituted C₁ to C₁₀ alkyl).

The term “alkyl” is used herein to refer to both straight- and branched-chain saturated aliphatic hydrocarbon groups. In one embodiment, an alkyl group has 1 to about 30 carbon atoms (i.e., C₁, C₂, C₃, C₄, C₅ C₆, C₇, C₈, C₉, or C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or C₃₀). In a further embodiment, an alkyl group has 1 to about 10 carbon atoms (i.e., C₁, C₂, C₃, C₄, C₅ C₆, C₇, C₈, C₉, or C₁₀). In another embodiment, an alkyl group has 4 to about 10 carbon atoms (i.e., C₄, C₅, C₆, C₇, C₈, C₉, or C₁₀). In a further embodiment, an alkyl group has 5 to about 10 carbon atoms (i.e., C₅, C₆, C₇, C₈, C₉, or CO.

The term “cycloalkyl” is used herein to refer to cyclic, saturated aliphatic hydrocarbon groups. In one embodiment, a cycloalkyl group has 4 to about 10 carbon atoms (i.e., C₄, C₅, C₆, C₇, C₈, C₉, or C₁₀). In another embodiment, a cycloalkyl group has 5 to about 10 carbon atoms (i.e., C₅, C₆, C₇, C₈, C₉, or C₁₀).

The term “alkenyl” is used herein to refer to both straight- and branched-chain alkyl groups having one or more carbon-carbon double bonds. In one embodiment, an alkenyl group has 2 to about 30 carbon atoms (i.e., C₁, C₂, C₃, C₄, C₅ C₆, C₇, C₈, C₉, or C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or C₃₀). In a further embodiment, an alkenyl group has 2 to about 10 carbon atoms (i.e., C₂, C₃, C₄, C₅ C₆, C₇, C₈, C₉, or C₁₀). In another embodiment, an alkenyl group has 4 to about 10 carbon atoms (i.e., C₄, C₅, C₆, C₇, C₈, C₉, or C₁₀). In a further embodiment, an alkenyl group has 5 to about 10 carbon atoms (i.e., C₅, C₆, C₇, C₈, C₉, or C₁₀). In another embodiment, an alkenyl group has 1 or 2 carbon-carbon double bonds.

The term “alkynyl” is used herein to refer to both straight- and branched-chain alkyl groups having one or more carbon-carbon triple bonds. In one embodiment, an alkynyl group has 2 to about 30 carbon atoms (i.e., C₁, C₂, C₃, C₄, C₅ C₆, C₇, C₈, C₉, or C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or C₃₀). In a further embodiment, an alkynyl group has 2 to about 10 carbon atoms (i.e., C₂, C₃, C₄, C₅ C₆, C₇, C₈, C₉, or C₁₀). In another embodiment, an alkynyl group has 4 to about 10 carbon atoms (i.e., C₄, C₅, C₆, C₇, C₈, C₉, or C₁₀). In a further embodiment, an alkynyl group has 5 to about 10 carbon atoms (i.e., C₅, C₆, C₇, C₈, C₉, or C₁₀). In another embodiment, an alkynyl group contains 1 or 2 carbon-carbon triple bonds.

The term “halogen” as used herein refers to Cl, Br, F, or I groups.

The term “aryl” as used herein refers to an aromatic, carbocyclic system, e.g., of about 6 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of about 6 to about 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 1 to 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 1 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “thioaryl” as used herein refers to the S(aryl) group, where the point of attachment is through the sulfur-atom and the aryl group can be substituted as noted herein. The term “alkoxy” as used herein refers to the O(alkyl) group, where the point of attachment is through the oxygen-atom and the alkyl group can be substituted as noted herein. The term “thioalkyl” as used herein refers to the S(alkyl) group, where the point of attachment is through the sulfur-atom and the alkyl group can be substituted as noted herein.

The term “hydroxyalkyl” refers to -(alkyl)OH, where the point of attachment is group through the alkyl group and the alkyl groups is defined above.

The term “alkylcarbonyl” as used herein refers to the C()(alkyl) group, wherein the point of attachment is through the carbon-atom and the alkyl group can be substituted as noted herein.

The term “alkylcarboxy” as used herein refers to the C(O)O(alkyl) group, wherein the point of attachment is through the carbon-atom and the alkyl group can be substituted as noted herein.

The term “test molecule” as used herein in screens to identify the TAAR antagonists can refer to any known or novel molecule for testing as a malodor inhibitor or a molecule to modulate malodor perception. Such molecules may typically be found in known libraries of molecules, including those that have been pre-screened e.g., for safe use in animals. Suitable test molecules may be found, for example, in AMES library and may be readily obtained from vendors such as Otava, TimTec, Inc., Chem Bridge Corp., etc. See e.g., Bhal et al, 2007 Mol. Pharmaceutics, 4(4):556-560. The test molecules/TAAR antagonists identified by the methods of this invention may be chemical compounds, small molecules, nucleic acid sequences, such as cDNAs, or peptides or polypeptides, which reduce, inhibit or block the perception of malodor. In one embodiment, the test compounds interact with the TAAR proteins. In one example, the TAAR protein is human TAAR5.

Depending upon their intended use, the TAAR antagonist compounds of Table I and their derivatives or mixtures can be assessed for fitness for their intended purpose by determining their GRAS and OSHA status. One of skill in the art may obtain these characteristics.

### II. COMPOSITIONS CONTAINING TAAR ANTAGONIST COMPOUNDS

The TAAR antagonist compounds described herein substantially eliminate or reduce the perception of malodors which activate the TAARs. In another embodiment, the TAAR antagonist compounds prevent the formation of such malodors. In certain embodiments, the TAAR antagonist compound(s) are provided in a composition with other components to form useful compositions. In one embodiment the TAAR antagonist in the composition is phenethyl benzoate or a derivative thereof. In another embodiment, the composition contains trans-2-nonen-1-ol or a derivative thereof and a carrier. In another composition, the TAAR antagonist compound is isoeugenyl phenlacetate or a derivative thereof. In yet another useful composition the compound is tributyl-2-acetylcitrate or a derivative thereof. In still another useful composition, the TAAR antagonist compound is bis(2-methyl-3-furyl)disulfide or a derivative thereof. Still other useful compositions contain one or more of the other compounds of Table I or derivatives, mixtures, racemers, enantiomers, or any combination thereof.

In general, such useful compositions can fall into a category such as a consumer product, e.g., foodstuff or ingestible compositions, a medicinal or vitamin composition, household chemical products, or an industrial chemical product. Useful compositions containing at least one TAAR antagonist compound described herein can be in solid, powder, granular composition, an aqueous liquid, an oil-based liquid, a gel, an emulsion or semi-solid form, depending upon the particular use. As one example, a useful composition containing a TAAR antagonist compound can be one or more of the compositions identified in U.S. Pat. No. 8,506,943, or US Patent Publication No. 2013026664, incorporated by reference herein.

“Consumer products” include without limitation, conventional products designed for infant care, pet care, first aide products, beauty products, home care products for treating surfaces, e.g., fabrics, upholstery, rugs, bath and kitchen appliances, feminine hygiene, ingestible vitamins, supplements or foodstuff. Such products include but are not limited to products for bleaching, coloring, dyeing, conditioning, shampooing, styling; deodorants and antiperspirants; personal cleansing; cosmetics; skin care creams, lotions, and other topically applied products for consumer use; and shaving products, cleaning products, air fresheners, car fresheners, dishwashing, laundry products, hard surface cleaning and/or treatment, and other cleaning for consumer or institutional use; tissue and paper products, toothpastes, tooth gels, tooth rinses, denture adhesives, tooth whitening; cough and cold remedies, pain relievers, processed food products.

In one embodiment, the TAAR antagonist compound is used with other components to form a room deodorizer or neutralizer composition. Such a product may be in the form of a spray, an aerosol, a diffuser, a wick, a plug-in device, a candle, a wax, a sachet powder, a dry spray or a gel. In still other embodiments, the TAAR antagonist compound may be added to personal care products, such as deodorants, antiperspirants, foot sprays, hair sprays, or disinfectants for application to skin or to non-biological surfaces. Still other embodiments of compositions to which the TAAR antagonist compounds may be added are laundry detergents, fabric softeners, dryer sheets, or other types of fabric sprays. In other embodiments, these compounds can be added to a variety of household cleaning compositions for hard surfaces, such as bathroom and kitchen fixtures, waste bins, fixtures, etc.

Additionally, such compounds may be added to industrial compositions designed to eliminate or reduce malodors that bind TAARs. Among such compositions are metal finishing fluids, hydraulic fluids, paper-treating fluids, sewage treatment fluids, cleaners or polishers or fresheners useful on boats, e.g., to decrease “fishy” odors, etc.

The type of composition containing the TAAR antagonist compound will determine the carrier with which the compound is associated. In one embodiment, the term “carrier” refers to an aqueous solution or aqueous solvent into which the TAAR antagonist compound is admixed. In another embodiment, the carrier is an oil-based fluid or emulsion into which the TAAR antagonist compound is admixed. In such embodiments, the fluid compositions can contain propellants to aerosolize the composition. In such applications in which the compound is included in a composition which may be inhaled, those compounds of Table I that are OSHA approved for exposure in air, e.g., phenethyl benzoate and trans-2-nonen-1-ol, are included in the composition. One of skill in the art may readily determine the OSHA status of the compounds of Table I.

In still another embodiment, the “carrier” refers to a substrate which is coated with or impregnated with the compound, e.g., a fabric, wallpaper, wallboard, dryer sheets, personal care products, such as diapers, tampons, clothing, and the like. In still other embodiments, the carrier can be a powdery mixture containing other components for dilution. As one example, the compound may be impregnated in wrapping materials used in the food industry for wrapping fish or other foods having a strong odor, or for packaging and bags and other containers for the disposal of decaying biological waste materials, among others.

In additional compositions intended for mammalian, human or animal treatment or ingestion, the TAAR antagonist compound must be safe for application to skin or epithelial membranes or safe for ingestion. In one embodiment, the compound is added to liquid medicines to ameliorate the malodor associated with the medicine. In another embodiment, the compound is added to an ingestible foodstuff or vitamin or additive. For example, tributyl-2-acetylcitrate and isoeugenyl phenylacetate are useful in such compositions as both are GRAS approved for use in foods. Any other of the compounds of Table I, which are GRAS approved may be useful in similar compositions. One of skill in the art may readily identify those compounds in Table I which are GRAS approved for use in ingestible compositions. For example, a TAAR antagonist compound may be admixed with fish oil in dietary supplement pills to reduce fishy or other strong odors or with any number of products having strong odors. In still another embodiment, the TAAR antagonist compounds are included in produce cleaning solutions or sprays.

The identities of the additional components forming the products discussed above may be selected by one of skill in the art from conventional components for similar uses. Such components include without limitation, propellants, carriers, surfactants (nonionic, anionic, cationic, amphoteric), solvents, corrosion inhibitors, oxidation inhibitors, defoamers, thickeners, film-forming agents, emulsifiers, water, chelating agents, pH adjusting agents, fragrances, plant extracts, herbal medicine components, alcohols, esters, long-chain fatty acids, amino acids, organic amines, antibacterials, antiseptics, antifungal agents, sugars, salts, dyes, dispersants, enzymes, and other ingredients suitable to the composition. See, e.g., U.S. Pat. No. 6,592,813, US Published Application No. 2014/0216492, and US Published Application No. 20120219520 incorporated by reference herein, for discussion of other components included with neutralizing sprays, gels or other compositions. See, e.g., U.S. Pat. Nos. 8,629,092 and 8,461,089 for other ingredients suitable for use in cleaning compositions. See, e.g., U.S. Pat. No. 8,395,012, US Published application No. 20130266642, U.S. Pat. No. 8,101,124 or US Published Application No. 20120219520 for other ingredients suitable for use in odor controlling films or air fresheners or fabrics. See, also, for example, U.S. Pat. No. 7,666,685 for other ingredients suitable for use in mouthwashes. All documents are incorporated by reference herein.

### III. METHODS OF USE OF TAAR ANTAGONIST COMPOUNDS

In one aspect, a method for blocking, inhibiting, counteracting or reducing the perception of malodor by a mammalian subject comprises blocking TAAR olfactory receptors of a subject with a TAAR antagonist composition or compound as described herein. As discussed above, where the subject is a human subject and the compositions are intended for human contact, inhalation or ingestion, the antagonists are designed to reduce the response of the TAAR receptor to the malodor. In one embodiment, the TAAR receptor is human TAAR5. In other embodiments, the TAAR receptor is any of the known or identified human TAARs. Where the subject is an animal subject, i.e., pets, farm animals, etc., and the composition is intended for animal contact, inhalation or ingestion, the antagonists are selected to compete with the malodor target for an animal TAAR receptor. Obviously for most industrial and household uses, the human TAAR receptors are more appropriate targets.

Thus, in one embodiment, a method of using the TAAR antagonist compounds is by admixing with, or introducing to, a product containing a malodor that stimulates a TAAR an effective amount of a the TAAR antagonists compound. The TAAR antagonist compound can be any one or more of the compounds of Table I, any one or more derivatives of such compounds, or any mixture or combination of compounds or derivatives. The composition that contains the malodor can be any foodstuff, industrial composition or household compositions that present unpleasant odors that stimulate a TAAR. In one embodiment the unpleasant odor is the fishy odor that is found to stimulate TAAR5. However, other TAARs may be targeted by these compositions. Such malodor containing compositions can be a gas, solid, semi-solid or liquid product. Such products can be a paint, a cleanser, a household chemical, or an industrial chemical.

In another embodiment, a method of using the TAAR antagonist compounds is by admixing with, or introducing to, a deodorizing or cleaning product intended to reduce a malodor that stimulates a TAAR. In this example, the malodor is not present in the composition but is present in the air, surface or other substrate requiring cleaning or deodorizing. The product in question may or may not contain other malodors, but application of the product by aerosolizing or applying it to a surface is intended to reduce the sensitivity of subjects exposed to that malodor. The air or surface that contains the malodor can be any foodstuff, industrial composition or household compositions that present unpleasant odors that stimulate a TAAR. In one embodiment the unpleasant odor is the fishy odor that is found to stimulate TAAR5. However, other TAARs may be targeted by these compositions. Such malodor combating compositions can be a gas, solid, semi-solid or liquid product. Such products can be a cleanser, a household deodorizer, an air freshener, a vegetable cleaning composition, etc.

In still another embodiment, a method of using the TAAR antagonist compound described herein involves admixing the compound as an additive to an ingestible or topical composition, e.g., medicine, vitamin, supplement (e.g., fish oil tablets) or foodstuff that contains a malodor that stimulates a TAAR. In yet another embodiment, a method of using the TAAR antagonist compound described herein involves administering or applying an effective amount of a TAAR antagonist prior to or concurrently with use of a composition containing a malodor that stimulates a TAAR.

In the methods of making the compositions containing the TAAR antagonist compounds, the compounds are added to each composition or applied to each substrate in an amount that is effective to reduce or eliminate the sensitivity of a subject (mammalian, human or animal) to a particular malodor while the subject is in contact with the malodor. Therefore, the amount of TAAR antagonist compound in any composition will depend upon its intended use, whether or not it will be ingested and metabolized, or whether it is intended to be deposited on a surface, the nature of the carrier, and similar practical aspects. It is anticipated that one of ordinary skill in the art of preparing the given composition is capable of adjusting the amount of the compound to suit the use. In one embodiment, a personal care composition contains a TAAR antagonist compound in an amount ranging from about 0.001% to about 10% by weight of the product, including at least 0.01%, 0.1%, 1%, 5% or 10% or more, and further including any percentage or fractional percentage between any two of the numbers identified. In another embodiment, an industrial product or cleaner for hard surfaces contains a TAAR antagonist compound in an amount ranging from about 0.01% to about 20% by weight of the product. In yet another embodiment, an industrial composition includes at least 0.01%, 0.1%, 1%, 5%, 10%, 15%, 20% or more, and further including any percentage or fractional percentage between any two of the numbers identified.

### IV. METHODS OF IDENTIFYING MALODOR BLOCKERS

In another aspect, a method for identifying or screening malodor blocking molecules, compounds or compositions involves screening for the effect of a test molecule, compound or mixture of compounds on the expression or activity of a selected trace amine-associated receptor (TAAR) protein in a cell-based assay. In one embodiment, this method involves contacting a test molecule with a mammalian cell or cell line that expresses a TAAR protein, e.g., the TAAR5 protein, under in vitro culture conditions.

Such cell based assays may employ a mammalian cell or cell lines that naturally express the desired TAAR protein or cells or cell lines that have been manipulated to express a TAAR protein that the cell does not ordinarily express. For example, a heterologous cell expressing the TAAR protein may be a human kidney cell (HEK) cell that is genetically engineered to express the desired TAAR protein. Suitable cells may be selected and manipulated to express the desired TAAR protein. Selected cells or cell lines can be A549, WEHI, 3T3, 10T1/2, HEK 293 cells, PERC6, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell.

The expression level or functional activity of the TAAR by the contacted cell or cell line can be detected or measured in the presence and absence of the test compound and/or a control. By “expression level” is meant the quantitative expression of the nucleotide sequence (e.g., mRNA) of a desired protein encoding sequence (e.g., TAAR5) or the quantitative expression of the desired protein itself. By “functional activity” is meant the expected normal activity of the TAAR mRNA or protein or channel when expressed in a cell.

In one embodiment, the binding between the test molecule and the TAAR receptor is detected by a binding assay employing a label bound to the test molecule. Conventional binding assays employing conventional labels may be employed in this assay. Conventional detectable labels may include an enzyme, a fluorochrome, a luminescent or chemi-luminescent material, or a radioactive material. Methods of associating such labels with a test molecule are known to those of skill in the art. Thus to detect a TAAR binding to the test molecule, in one embodiment, the mammalian cell or cell line that expresses TAAR is contacted with a test molecule or compound that has been labeled with a detectable label. The cells are then washed to remove any non-bound labeled test molecule or compound. The cells are then analyzed to detect the presence of the bound test molecule-TAAR complex by detecting the detectable label.

The method may further employ a counter-screen assay on the test molecule to exclude non-specific activity, e.g., to exclude non-TAAR binding activity of the test molecule in the cell or cell line tested. A counter-screen assay is performed concurrently in which the same method steps are performed using a mammalian cell or cell line does not express the TAAR. The counter-screen assay acts as the negative control and allows for the detection of test compounds that bind the expressed TAAR.

Additionally, the method may further include screening the test molecule by subjecting the test molecule to an animal physiological response, electrophysiological response, or behavioral assays to determine the ability of the test molecule to block the perception of malodor in the presence of such a malodor.

In one embodiment, detection of a test compound or molecule that binds the TAAR protein provides the information that the molecule can inhibit, reduce or block malodor perception by the TAAR. Thus, binding is an indication of a malodor blocker or reducer. In another embodiment, a decrease in protein expression or functional activity of the TAAR protein by the cell or cell line contacted with the test molecule compared with the level of expression or activity in the presence of a positive or negative control permits the identification of the test molecule as one that can inhibit, reduce or block malodor perception.

In still another embodiment, a second molecule or agonist known to bind or activate the activity or expression of the TAAR protein is used as a comparative control. A cell based assay is used to determine whether the test compound in contact with the cells expressing the TAAR binds to the TAAR (or otherwise inhibits interaction between the TAAR and the agonist) preferentially in comparison to the second compound, i.e., whether the test compound competitively displaces the second compound. In one embodiment, TMA, which is known to interact with TAAR5, is useful as a second molecule/agonist in such a screen. If the test compound can be detected or measured to displace the TMA in binding the TAAR or in effecting its activity or expression, such displacement is an indication that the test compound is a malodor blocker, inhibitor or reducer. A cell-based assay for such screening and the methods used for detection and measurement are discussed in detail in the examples below. One of skill in the art may readily adapt other methods, vectors and components of a cell-based screen to accomplish such identification of malodor blockers or inhibitors, in view of the teachings provided herein and the teachings of the documents cited below in the examples, which are all incorporated by reference in this specification.

### V. EXAMPLES

Examples 1-2 demonstrate the identification of certain compounds as antagonists for malodors. Example 3 illustrates an air freshener product containing a malodor antagonist. The following examples are illustrative of the claimed invention.

### Example 1: Identifying Agonists and Antagonists of Trace Amine-Associated Receptors

We screened a TAAR of interest, TAAR5, with the agonist TMA added at the EC80 concentration, against a proprietary small-molecule library to identify antagonists. TAAR open reading frames were amplified from pooled human genomic DNA using Phusion polymerase and subcloned into a pCI expression vector (Promega) containing the first 20 residues of human rhodopsin (Rho tag). The sequences of the cloned receptors were verified by sequencing (3730xl Sequencer, ABI Biosystems). We used the Dual-Glo™ Luciferase Assay System (Promega) to measure receptor responses as previously described in Trimmer, C. et al 2014 High-throughput Analysis of Mammalian Olfactory Receptors: Measurement of Receptor Activation via Luciferase Activity. Journal of Visualized Experiments, (88). doi:10.3791/51640. Hana3A cells were transfected with 5 ng/well of RTP1S, 5 ng/well of pRL-SV40, 10 ng/well of CRE-luciferase, 2.5 ng/well of M3, and 5 ng/well of odorant receptor. 1M odorant stocks were diluted in DMSO. 24 hours following transfection, transfection media was removed and replaced with the appropriate concentration of odor diluted from the 1M stocks in CD293 (Gibco). Four hours following odor stimulation luminescence was measured using a Synergy 2 plate reader (Biotek). All luminescence values were divided by the Renilla Luciferase activity to control for transfection efficiency in a given well.

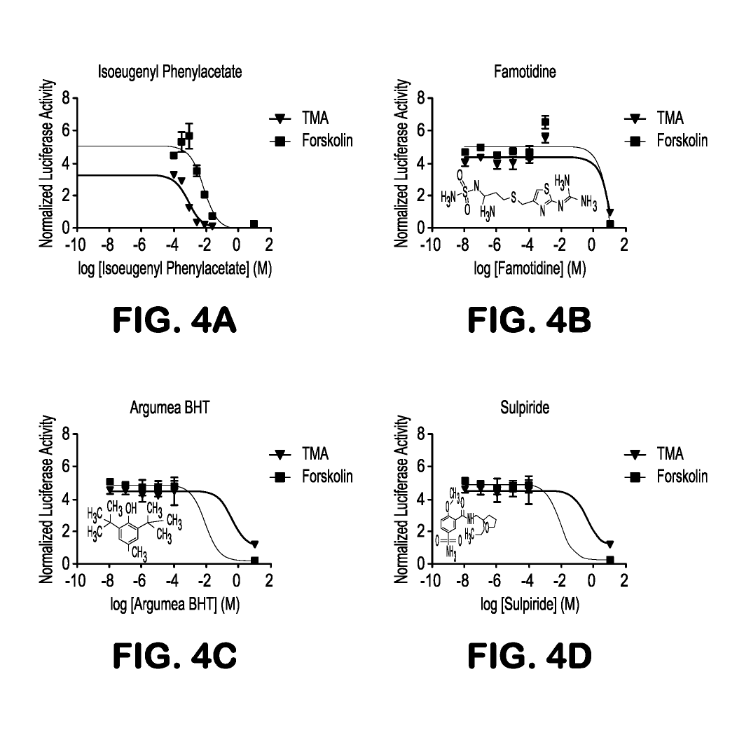

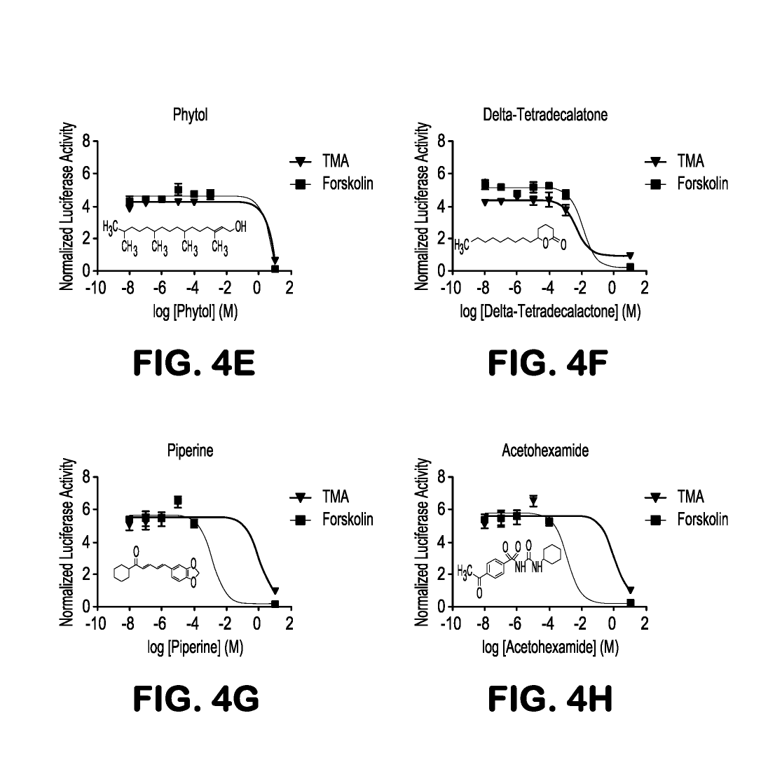

We completed an antagonist screen of a proprietary library of odors, including the 52 odorous compounds identified in FIG. 4 at a concentration of 30 μM. The top twelve hits from the antagonist screen, i.e., the first 12 compounds identified in FIG. 4, were selected for follow up experiments.

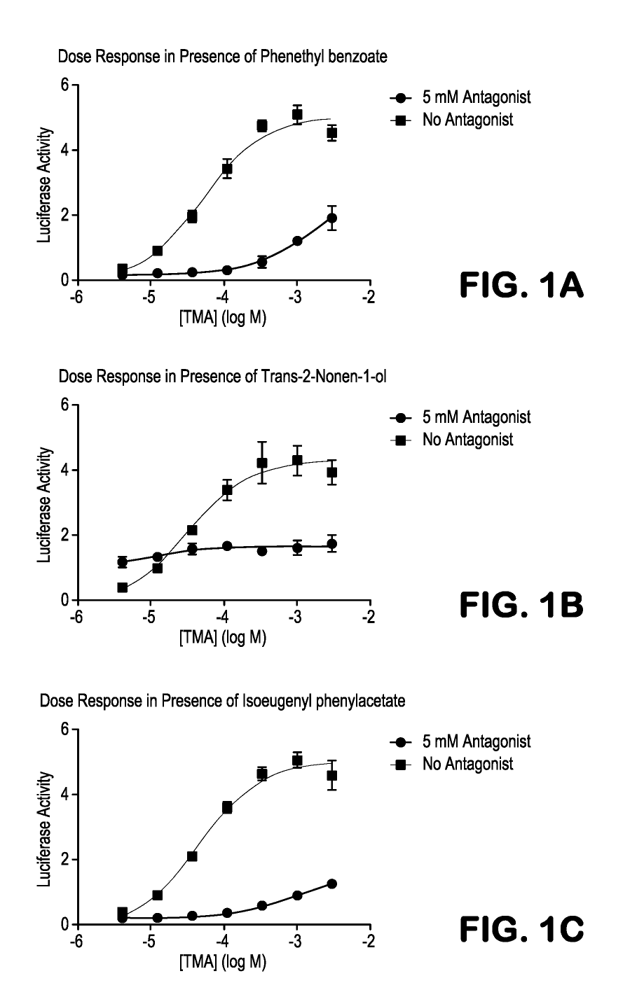

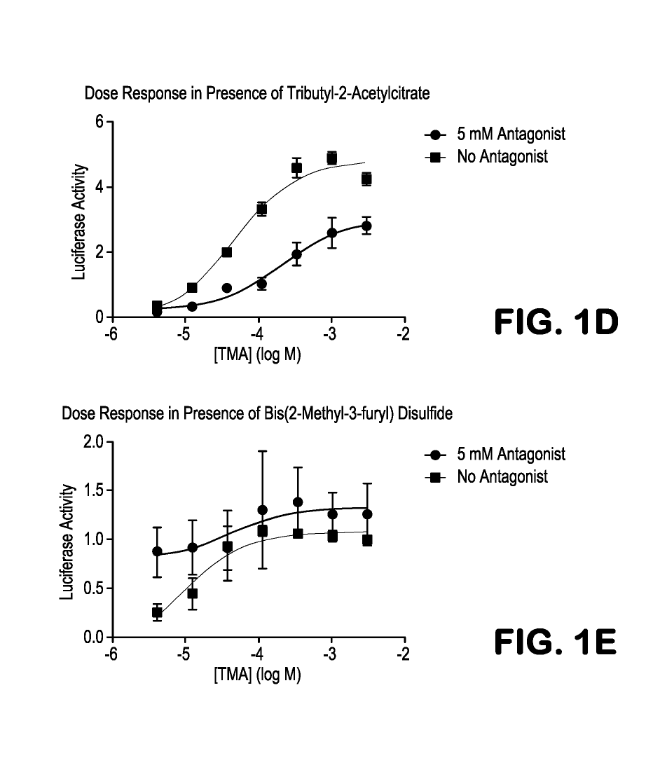

In a secondary experiment, we ran dose dependent inhibition responses for the top twelve antagonists, i.e., the top 12 compounds identified in FIG. 4. TMA was kept at the EC80 (500 μM) and the twelve antagonists were diluted in a dose response with the highest concentration at 1000 μM. The top five candidate compounds, i.e., phenethyl benzoate, trans-2-nonen-1-ol, isoeugenyl phenlacetate, tributyl-2-acetylcitrate, bis(2-methyl-3-furyl)disulfide, were rescreened using an antagonist dose response at 2500 μM. We determined the IC80 values for those five antagonists. Using those IC80 values, we completed a 3-fold dilution dose response of TMA, starting at 3000 μM, in the presence of our five candidate antagonists. The results are demonstrated in FIGS. 1A-1E. It was difficult to determine if the five antagonists were competitive or non-competitive inhibitors using the very high IC80 concentrations of the antagonists.

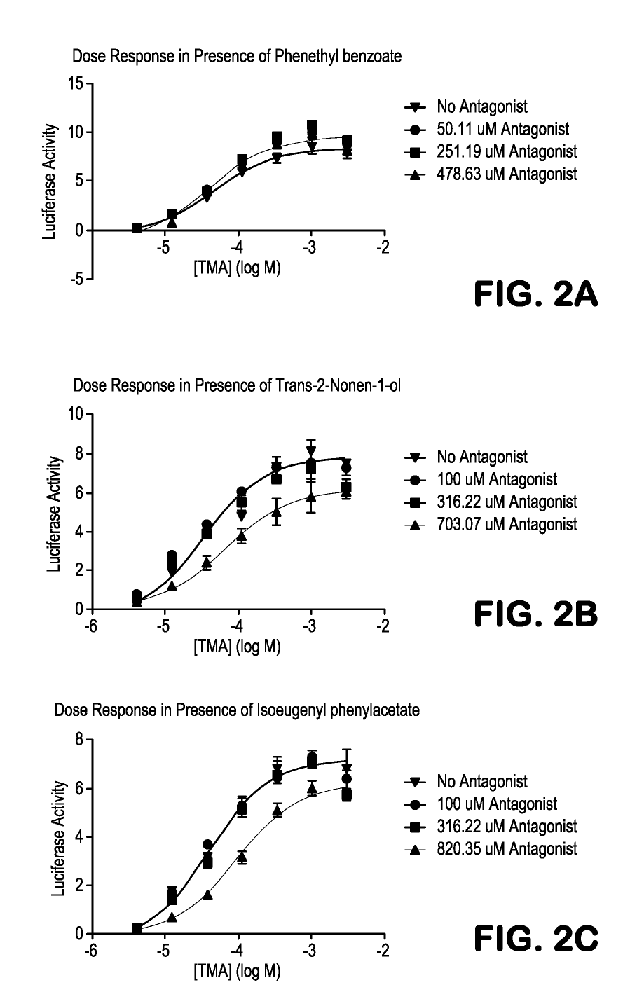

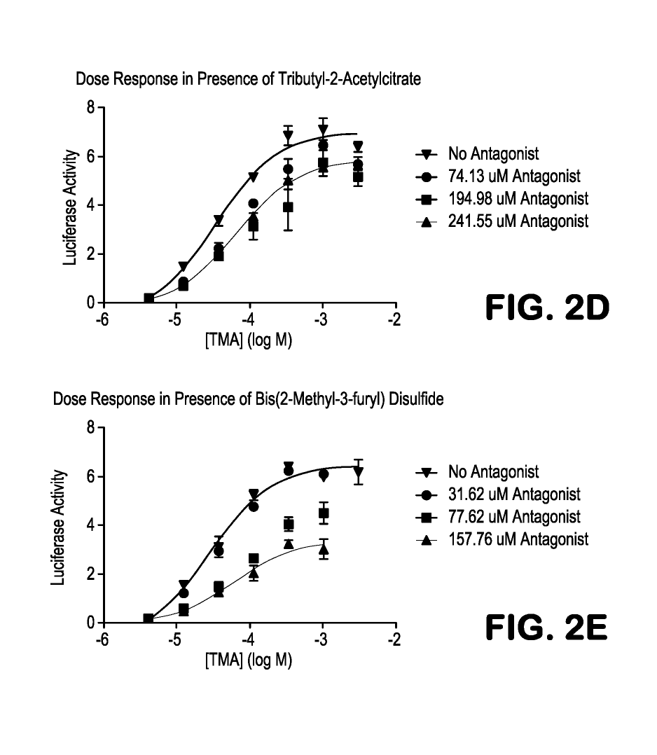

In another experiment, we evaluated dose responses of TMA in the presence of the antagonist at their IC10, IC30 and IC50 concentrations. These concentrations are reported in the attached FIGS. 2A-2E, which demonstrate the results of this experiment.

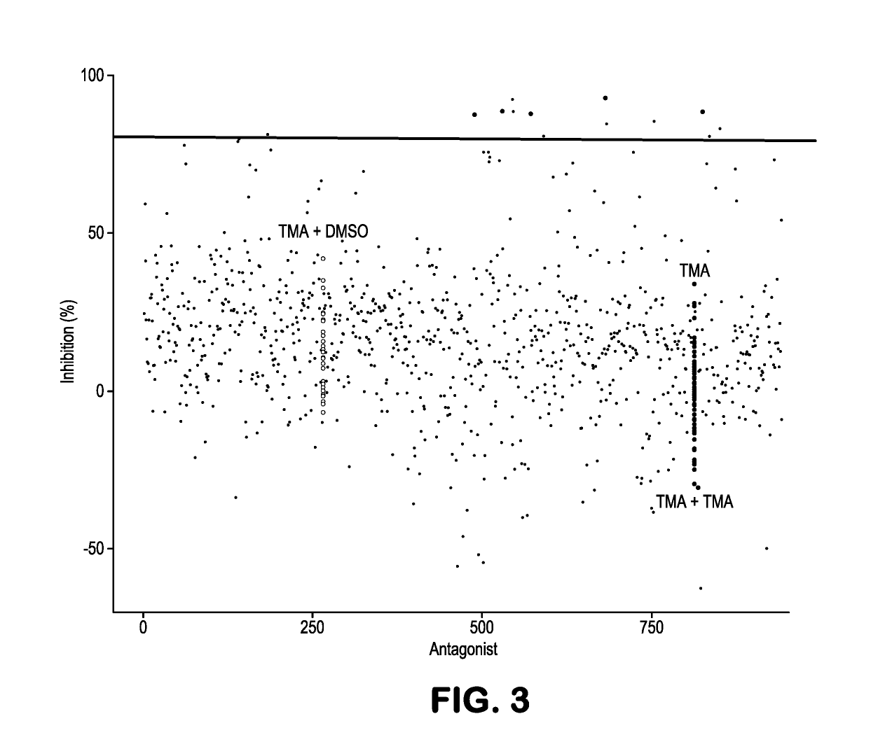

FIG. 3 shows the inhibition percentage plot from the original antagonist screen with the five antagonists highlighted in green along with the line representing 80% inhibition.

In another experiment, we examined physicochemically similar odorants to determine if they functioned as antagonists. We generated 4300 physicochemical descriptors using the Dragon software (Talete, Milan, Italy) and calculated the pairwise correlation between 853 monomolecular odorants. The seven most similar molecules showed no antagonism of the receptor (FIGS. 4A-4H).

### Example 2: Evaluation of TAAR Agonists and Antagonists at the Human Psychophysical Level

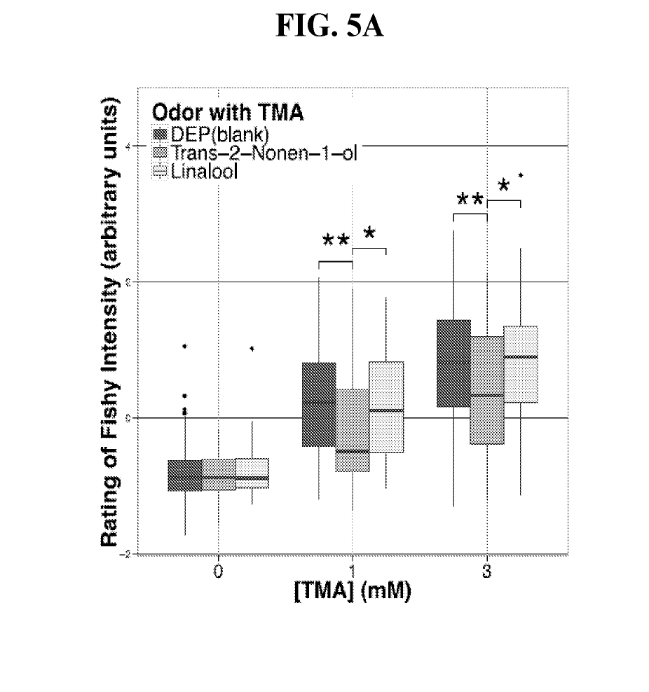

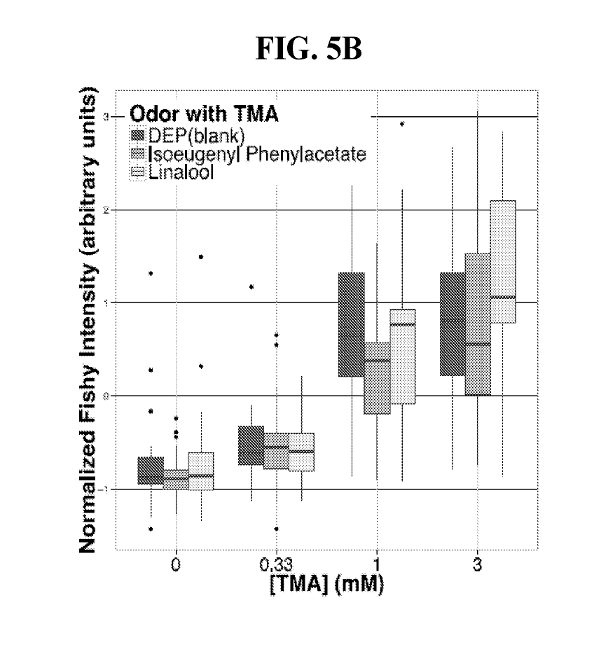

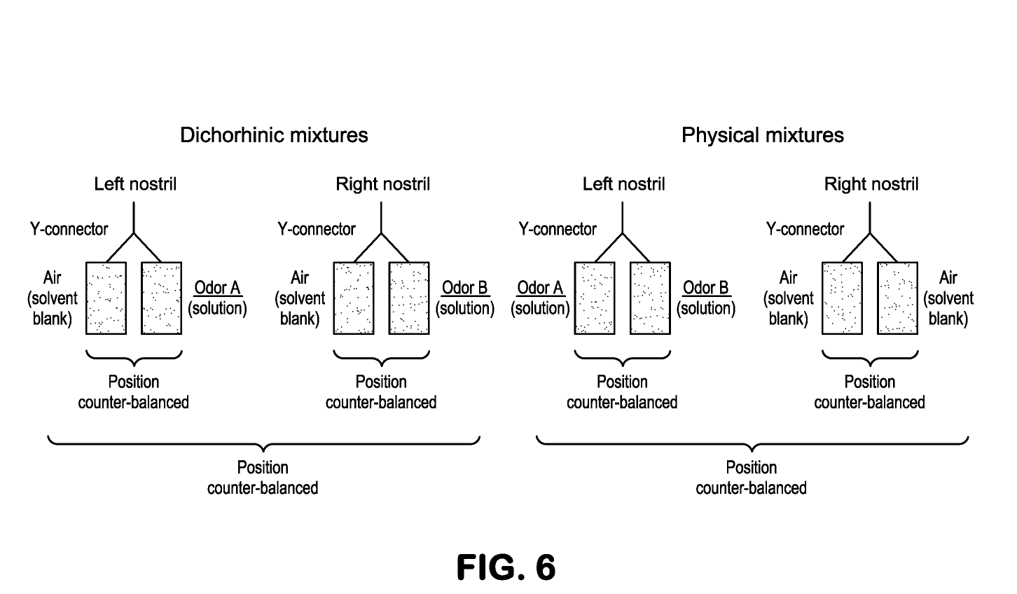

We evaluated the efficacy of the in vitro antagonists on human perception. We asked 10 (FIG. 5A) or 29 (FIG. 5B) subjects to rate how “fishy” and how “pleasant” an odor was. The odors were delivered to the subject using an air-dilution olfactometer (Lundström, J. N., et al., 2010 International Journal of Psychophysiology, 78(2), 179-189; doi:10.1016/j.ijpsycho.2010.07.007) and each trial consisted of one concentration of trimethylamine (either 3 mM, 1 mM, 0.33 mM, or a water blank) combined with either an antagonist or a solvent blank. Subjects used a general labeled magnitude scale to rate how “fishy” the odor smelled and a scale from −10 to +10 to rate the pleasantness.

The antagonists and control odors were intensity-balanced to match the 1 mM concentration of TMA. Trans-2-nonen-1-ol was diluted in Diethyl phthalate (DEP); TMA was diluted in water with NaOH added; Linalool was diluted in DEP. In all trials subjects received air from jars containing water and DEP so that solvent presence could not be used as a cue to the presence of the test odor. Subjects (n=39) successfully rated increasing concentrations of TMA as increasingly fishy, and adding the control odor linalool had no effect on these fishy ratings. Trans-2-nonen-1-ol, however, significantly decreased the perceived fishy rating compared to the solvent control and TMA combined with linalool, which did not show in vitro antagonism (FIG. 5A). Isoeugenyl Phenylacetate showed a similar pattern (FIG. 5B).

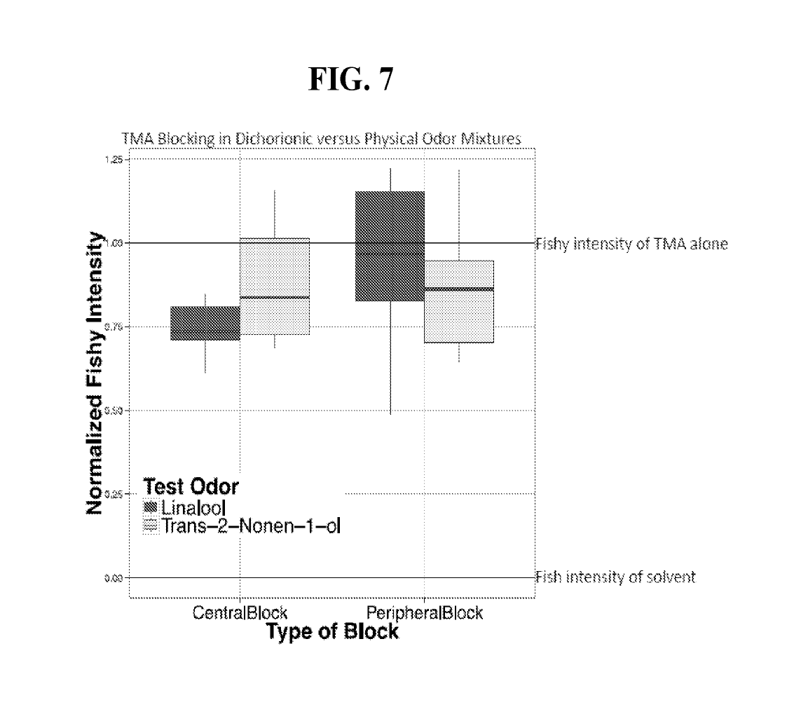

Mixture suppression has both a central and peripheral component (Laing, D., & Willcox, M. (1987). An investigation of the mechanisms of odor suppression using physical and dichorhinic mixtures. Behavioural Brain Research, 26(2-3), 79-87. http://doi.org/10.1016/0166-4328(87)90157-4). We hypothesized that the antagonists identified in cell culture would show an increased peripheral suppression relative to odorants that do not antagonize hTAAR5. To test this we carried out split-nostril experiments using stimuli in glass jars. We compared TMA-alone, TMA mixed with the probe odorant in the same nostril (physical mixture), and TMA mixed with the probe odorant in different nostrils (dichorhinic mixture) (FIG. 7). In the dichorhinic mixture, due to the anatomical separation between nostrils, there is no peripheral antagonism at the receptor site as long as the flow does not reverse in the nostril. Subjects were instructed to make intensity judgements during inhalation to avoid retronasal contamination across nostrils. This allowed us to separate central and peripheral components of the mixture suppression. We found that trans-2-nonen-1-ol had a larger peripheral component of mixture suppression than the control odorant linalool.

### Example 3: Exemplary Air Freshener Product

The following ingredients form an air freshener product containing a TAAR antagonist compound. An air freshener product is designed by combining a selected fragrance, e.g., orange concentrate, peach apple crisp, or other essential oil at 3% w/w with an aerosolizing formulation such as sodium di-2-ethylhexylsulfosuccinate (Cytec) at 1.5% w/w, methyl carbitol (Dow Chemical) at 10.0% w/w, butyl carbitol (Dow Chemical) at 20% w/w, water at 63.5% 2/2 and a TAAR antagonist compound of FIG. 4, e.g., phenethyl benzoate or trans-2-nonen-1-ol or isoeugenyl phenylacetate or tributyl-2-acetylcitrate or bis(2-methyl-3-furyl)disulfide, or derivatives or combinations, at 2% w/w. This formula can be introduced into a device or dispenser such as that described in US Patent Publication No. US2012/0097754, incorporated by reference herein. Optionally, additional components suitable for such air freshener formulations, as described in the reference may be included.

All publications and documents cited in this specification and priority U.S. provisional application No. 62/112,379 are incorporated herein by reference herein. Some of these documents are incorporated to provide instruction on methods or components available to one of skill in the art, which may be used according to the teachings of this specification. While the invention has been described with reference to particular embodiments, it will be appreciated that modifications can be made without departing from the spirit of the invention. Such modifications are intended to fall within the scope of the appended claims.

1. A composition for blocking, inhibiting or reducing the perception of malodor by sensory receptors of a mammalian subject comprising an effective amount of a compound of Table I or a derivative thereof in a carrier.

2. The composition according to claim 1, wherein the compound is trans-2-nonen-1-ol or a derivative thereof, or phenethyl benzoate or a derivative thereof, or isoeugenyl phenylacetate or a derivative thereof or tributyl-2-acetylcitrate or a derivative thereof, or bis(2-methyl-3-furyl)disulfide or a derivative thereof.

3-6. (canceled)

7. The composition according to claim 1, which comprises a foodstuff, a medicine, an industrial chemical product, a household chemical product, a paint, a cleanser, an air freshener, a food supplement, a vitamin, or a vitamin supplement.

8. The composition according to claim 1, which is an air freshener or deodorizer composition.

9. (canceled)

10. A method for blocking, inhibiting, counteracting or reducing the perception of malodor by a mammalian subject comprising blocking the TAAR olfactory receptors with a compound of Table I or a derivative thereof.

11. The method according to claim 10, wherein said blocking comprises admixing with, or introducing to, a product containing a malodor that stimulates a TAAR an effective amount of a compound of Table I or a derivative thereof.

12. The method according to claim 10, wherein said blocking comprises administering an effective amount of a compound of Table I or a derivative thereof with a composition containing a malodor that stimulates a TAAR.

13. The method according to claim 10, wherein said blocking comprises applying an effective amount of a compound of Table I or a derivative thereof prior to or concurrently with use of a composition containing a malodor that stimulates a TAAR.

14. The method according to claim 10, wherein said blocking comprises admixing with, or introducing to, a deodorizing or cleaning composition, an effective amount of a compound of Table I or a derivative thereof.

15. The method according to claim 10, wherein the subject is a human.

16. The method according to claim 10, wherein the TAAR receptor is human TAAR5.

17. The method according to claim 10, wherein the compound is trans-2-nonen-1-ol or a derivative thereof, phenethyl benzoate or a derivative thereof, isoeugenyl phenylacetate or a derivative thereof, tributyl-2-acetylcitrate or a derivative thereof or bis(2-methyl-3-furyl) disulfide or a derivative thereof.

18. The method according to claim 10, wherein the composition or product is a gas, solid or liquid product.

19. The method according to claim 18, wherein said product is a paint, a cleanser, a household chemical, an air freshener, an industrial chemical, a foodstuff, a food supplement, a vitamin, a vitamin supplement.

20. A method for identifying or screening malodor blocking molecules, compounds or compositions comprises contacting a mammalian cell or cell line that expresses a trace amine-associated receptor (TAAR) protein under in vitro culture conditions with a test molecule or compound; and detecting or measuring the expression level or functional activity of the TAAR protein in the contacted cell or cell line in the presence and absence of the test compound or a control.

21. The method according to claim 20, further comprising detecting binding of the TAAR protein by the test molecule.

22. The method according to claim 20, comprising measuring a decrease in protein expression or functional activity of the TAAR protein by the cell or cell line contacted with the test molecule over that of a positive or negative control cell or cell line.

23. The method according to claim 20, wherein the control is a second molecule known to bind or activate the activity or expression of the TAAR protein and wherein the ability of the test compound to displace the second molecule is detected or measured.

24. The method according to claim 20, wherein the TAAR is TAAR5.
